(19) United States
(12) Patent Application Publication
(10) Pub. No.: US 2022/0310247 A1
(43) Pub. Date: Sep. 29, 2022

(54) VIRTUAL REALITY THERAPEUTIC SYSTEMS

(71) Applicant: OXFORD VR LIMITED, Oxford (GB)

(72) Inventors: Daniel Freeman, Oxford (GB); Riana Patel, Oxford (GB); Jason Freeman, Oxford (GB); Christophe Faucherand, Oxford (GB); Mary Cordeiro, Oxford (GB); Aitor Rovira, Oxford (GB); Chung Yen Looi, Oxford (GB)

(21) Appl. No.: 17/619,698

(22) PCT Filed: Jun. 5, 2020
(86) PCT No.: PCT/EP2020/065707

§ 371 Date: Dec. 16, 2021

(30) Foreign Application Priority Data
Jun. 17, 2019 (GB) 1908647.9

(51) Int. Cl. G16H 40/67; G16H 20/70; A61B 5/16; A61B 5/0205

(52) U.S. Cl. CPC G16H 40/67; G16H 20/70; A61B 5/165; A61B 5/0205; A61B 5/02438

(57) ABSTRACT

A Virtual Reality (VR) therapeutic system for providing psychological therapy to a patient. The system includes at least a head-mounted display unit with a sound generation capability and a VR input device, and one or more computers. The system is configured to present a set of therapeutic scenarios to the patient in the virtual environment including an interaction task. A patient character interacts with a task character in the virtual environment; some of the time there is a coach character. The system determines a mental anxiety state parameter by measuring, in the virtual environment, one or more characteristics of the interaction of the patient character with the task character, monitors performance of the task, and in response to the mental anxiety state parameter and/or performance uses the coach character to provide VR feedback comprising one or both of a verbal prompt and a visual prompt in the virtual environment.

### CROSS REFERENCE TO RELATED APPLICATIONS

This application is the U.S. National Stage entry of International Application No. PCT/EP2020/065707 filed under the Patent Cooperation Treaty and having a filing date of Jun. 5, 2020, which claims priority to Great Britain Patent Application No. 1908647.9 having a filing date of Jun. 17, 2019, both of which are incorporated herein by reference.

### FIELD

This specification relates to virtual reality (VR) therapeutic systems for providing psychological therapy to a patient.

### BACKGROUND

As used herein a VR system includes a virtual reality headset, i.e. a head-mounted display unit, which provides a simulated and immersive virtual environment for a user.

Background prior art on the use of such technology can be found in U.S. Pat. No. 6,425,764; US2018/0190376; WO2019/036051; and Lancet Psychiatry 2018, 5: 625-32 Freeman et al.

### SUMMARY

This specification describes the use of VR systems for the treatment of psychological disorders. These are disorders common but the resources for treatment are often lacking.

Thus in a first aspect there is provided a virtual reality (VR) therapeutic system for providing psychological therapy to a patient. In implementations the VR therapeutic system comprises a VR system to provide a virtual environment and one or more computers coupled to or part of the VR system. The VR system may include at least a head-mounted display unit e.g. with a sound generation and/or detection capability, and one or more VR input devices e.g. a hand controller system to track the patient's hand movements such as a 3D mouse, glove, hand-held control device, or hand-tracking sensor(s). The VR system e.g. head-mounted display, may include a head motion tracking system. The one or more computers coupled to the VR system may be a workstation, or in a stand-alone VR system they may be incorporated into the head-mounted display unit (headset), or they may be remote from the headset e.g. in the cloud.

In implementations the one or more computers may be configured to present a set of therapeutic scenarios to the patient in the virtual environment using the VR system, e.g. to simulate a real-world scenario the patient finds difficult. Each therapeutic scenario may be defined by a spatial region in the virtual environment. Each therapeutic scenario may comprises at least one scenario task to be performed by the patient in the virtual environment by interacting with the VR system; this may be referred to as a patient action task as it involves action by the patient in the virtual environment. e.g. speaking, eating, or performing a mechanical action. Thus at least one of the scenario tasks may include at least an interaction task in which the patient character interacts with a task character in the virtual environment, or a non-verbal e.g. mechanical action task in which the patient character performs a non-verbal action in relation to one or more objects in the virtual environment.

The patient may be represented by a patient character in the virtual environment. Typically only a part of the patient is represented e.g. a hand and/or arm of the patient. At least some of the time, e.g. when a prompt is being delivered to the patient, the virtual environment may include a representation of a coach character. The representation may be a 3D representation of the coach character in the virtual environment or it may be a representation of an aspect of the coach character such as hand or footprints of the coach character.

The one or more computers may be further configured to determine a mental anxiety state parameter representing a mental anxiety state of the patient. This may be performed by measuring, in the virtual environment, one or more characteristics of the interaction of the patient character with the task character or the one or more objects. The one or more computers may be further configured to monitor performance of the scenario task in the virtual environment.

In response to one or both of non-performance of the scenario task, e.g. after an elapsed time, and a state of patient mental anxiety indicated by the mental anxiety state parameter the system may assist the patient in completion of the scenario task in the virtual environment by using the coach character to provide VR feedback. The VR feedback may comprise one or both of a verbal prompt, i.e. speech by the coach character, and a visual prompt provided in the virtual environment e.g. in response to detection of the state of patient mental anxiety. The prompt may comprise instructions or guidance for performing the task and/or may comprise spoken words e.g. for calming or encouraging the patient.

In some implementations the one or more computers may determine the mental anxiety state parameter by making one or more measurements in the virtual environment. The measurement in the virtual environment may involve determining a behaviour or characteristic of the patient character in the virtual environment. In implementations the measurement in the virtual environment does not comprise making a biometric measurement on the patient in the real world.

For example the one or more computers may measure a proximity of the patient character to the task character e.g. by reading their respective positions and determining a distance metric between the positions; or proximity to a task-related object in the virtual environment. Also or instead the VR system may include an eye tracking module and the one or more computers may measure i.e. determine an eye gaze direction in the virtual environment. For example in the case of an interaction with a task character this may be used to determine when the patient (character) is looking down e.g. at the feet of the task character, or looking away from the task character. Similarly the eye gaze direction may be used to determine when the gaze is averted from a difficult or distasteful task.

In another example the one or more computers may measure a time the patient character (i.e. patient) takes to respond to a verbalization of the task character, or a time the patient character (i.e. patient) takes to act in relation to the one or more objects. This may establish when the patient (character) is reluctant to perform a task or slow to respond. Some or all of these measurements may be used to determine or adjust a value of the mental anxiety state parameter, e.g. to increase (or decrease) a value of the parameter dependent upon a measurement. Also or instead the one or more computers may measure a body pose of the patient character in the virtual environment.

Also or instead the system may monitor performance of the scenario task e.g. by measuring an elapsed time since starting the task, or by determining whether or not the patient (character) speaks in response to a prompt e.g. a vocal prompt from the task character. In implementations the system may detect the presence or absence of patient speech but need not perform speech recognition.

In some implementations the VR therapeutic system may additionally make one or more real-world biometric measurements, and may additionally use these to determine the mental anxiety state parameter. For example one or more of the measurements, whether made in the real world or virtual environment, may be combined to determine the mental anxiety state parameter, for example using a weighted combination of (optionally scaled or normalized) measurements. Weights for the combination may be determined by routine experiment or automatically, e.g. using machine learning. A real-world biometric anxiety measurement may be determined using a handheld controller of the VR therapeutic system to connect to the patient. Such a real-world biometric anxiety measurement may comprise a measurement of e.g. galvanic skin response, heart rate, cortisol level or motion/activity level, as described further later. Another example of an anxiety measurement comprises a measurement of patient pupil size/dilation; this may be measured using an eye tracking or similar device. A further example of an anxiety measurement comprises a measurement of patient trembling, rapid hand movements, or more general body motion, e.g. made using the handheld controller(s). Such measurements may also be used where anxiety measurements are described later.

In some implementations a patient state neural network (or other machine learning subsystem) is provided to determine the mental anxiety state parameter. Inputs to the patient state neural network may comprise the one or more measurements in the virtual environment and/or the one or more real-world biometric measurements. The patient state neural network may directly output the mental anxiety state parameter, or the patient state neural network may output a set of scores or probability values, one for each of a set of classification categories, which may represent a degree of mental anxiety of the patient. These scores may be use to classify the patient as anxious or not anxious, and/or to grade patient anxiety into more than two categories. The patient state neural network may be trained using a supervised learning technique based on e.g. a patient's reported level of anxiety or on more objective measures established using existing techniques. For example anonymised data from previous patients e.g. with similar relevant scores. Alternatively the patient state neural network may be trained using a supervised learning technique. In implementations because the patient state neural network (classification) output determines the mental anxiety state parameter which is used to control the coach character it forms part of a closed-loop feedback (to the patient) system, and the mental anxiety state parameter may be used to adjust or control the experience of the patient in the VR environment, e.g. to calm the patient e.g. by spoken output, when patient anxiety is detected. The neural network output may also be used to predict the response of the patient, e.g. to the therapy or to individual scenarios, and/or to select scenarios or sets of scenarios with a high (highest) chance of positive outcome.

In some implementations the mental anxiety state parameter may be used to determine a state of patient mental anxiety by comparing a value of the mental anxiety state parameter with a first threshold value, which may be a fixed e.g. predetermined value or which may be variable dependent upon the patient and/or scenario and/or history (e.g. a running average). Also or instead a change in the value of the mental anxiety state parameter may be detected, e.g. a change of greater than a second threshold value, which again may be fixed or variable as previously described. Also or instead the value of the mental anxiety state parameter may be processed using an explicit or implicit pattern matching algorithm to determine the state of patient mental anxiety. For example the value of the mental anxiety state parameter may be provided to a trained neural network classifier, e.g. trained to provide anxious and not anxious classification outputs. Such as classifier may be trained by supervised learning using patients on whom other anxiety measurements have been made, e.g. heart rate and/or galvanic skin response (GSR) and/or cortisol level and/or motion or activity level. In other implementations the classifier is trained using unsupervised learning. Alternatively another machine learning-based classifier may be employed, such as a support vector machine (SVM) or random forest.

In implementations the feedback from the coach character includes speech, provided to the patient via the headset sound generation system. The VR system may include a patient mobile device configured to monitor one or both of patient activity and a measure of patient anxiety when the patient is in the real world and away from the virtual environment. For example the patient mobile device may comprise or consist of a wearable device such as a wristband configured, by means of a corresponding sensor, to make an anxiety measurement e.g. of heart rate and/or galvanic skin response (GSR) and/or cortisol level and/or patient motion or activity level (representing a degree of body motion of the patient, which may but need not include movement of the patient from one place to another). The patient mobile device may process the anxiety measurement e.g. heart rate/GSR/cortisol level/motion or activity level data to detect patient anxiety e.g. as previously described (by comparison with a threshold or threshold change, or by pattern matching). Cortisol level may be detected in sweat from the patient e.g. using the wearable sensor described in “Molecularly selective nanoporous membrane-based wearable organic electrochemical device for noninvasive cortisol sensing”, Parlak et al., Science Advances 20 Jul. 2018 Vol 4(7), DOI 10.1126/sciadv.aar2904. Also or instead the patient mobile device may detect a location of the device and use the location to identify when the patient is in a real-world scenario corresponding to one of the therapeutic scenarios in the virtual environment. The patient mobile device may, for example, comprise a mobile phone optionally coupled to a smart wristband.

The patient mobile device may thus use the monitored patient activity/anxiety to identify when the patient is in a stressful real world situation, a situation which may, but need not, correspond to one of the therapeutic scenarios. In response the patient mobile device may provide real world feedback i.e. feedback when the patient is in the real world and away from the virtual environment. The real world feedback may comprising a spoken audio output to the patient e.g. via headphone, earbuds or the like, in the voice the coach character uses in the virtual environment. For example the spoken audio output may comprise any words spoken using the voice of the coach character, and may include calming words and/or spoken instructions or encouragement.

The patient mobile device may communicate with the one or more computers via a remote server, e.g. in the cloud, which may also store/process patient-related data e.g. in an encrypted form. Optionally the one or more computers may be configured to capture patient data from the patient whilst the patient is in the therapeutic scenario in the virtual environment and provide this data e.g. in processed or encrypted form, to the patient mobile device e.g. via the remote server. The patient mobile device may be configured to use the captured data to provide the real world feedback. For example the captured data may include data relating to an anxiety level of the patient e.g. data derived from the mental anxiety state parameter, and this data may then be used to determine when the patient has a raised anxiety level in the real-world. Thus a real-world anxiety measure may be derived from the data for a specific patient or cohort of patients, e.g. by machine learning. Moreover the virtual world coach character, with whom the patient is familiar, may be transported to the real world to provide coaching in difficult real-world scenarios, typically by reproducing the voice of the coach character but potentially by reproducing a representation of the coach character.

The set of therapeutic scenarios may comprise therapeutic scenarios of the same type e.g. paying for an item, meeting a person, and so forth, but different levels of difficulty (see later), and/or they may comprise different types of therapeutic scenario. However in general each therapeutic scenario is configured to simulate a real-world scenario the patient finds difficult, and each has a set of increasing difficulty levels. In implementations the one or more computers are configured to provide a welcome room VR scenario (which is not one of the therapeutic scenarios) including the coach character. Within the welcome room the patient character may be enabled e.g. facilitated by the coach character, to select one of the therapeutic scenarios and/or a difficulty level for the selected therapeutic scenario. In some implementations the difficulty level is not less than a previously completed difficulty level for the (same type of) therapeutic scenario; in other implementations the patient may be permitted to retry scenarios of the same or lesser difficulty. Potentially progression through the levels of difficulty may also be based on or controlled by the patient's real world responses e.g. in similar scenarios.

In some implementations therapeutic scenarios of different difficulty level may be constructed procedurally, which may reduce the memory requirements of the system. For example the difficulty level of a selected therapeutic scenario may be changed by modifying one or more of a background noise level, a number of (task) characters in the selected therapeutic scenario, a duration of the therapeutic scenario/task, a location of the task in the virtual environment, and a behaviour of the task character(s).

For example to increase a difficulty level a task character may be configured to make direct eye contact with the patient character; and conversely to decrease a difficulty level a task character may be configured not to make direct eye contact with the patient character. Alternatively a frequency of eye contact of a task character with the patient character may be altered. Eye contact may be determined according to whether or not the gaze of a task character is directed towards the eyes of the patient character (i.e. is the patient character being observed); it may but need not require the patient character to look at the task character. Thus in some implementations a behaviour of the task character(s) is changed by changing a number of the task character(s) observing the patient character, e.g. by changing a parameter associated with each task character defining whether or not the task character is configured to observe the patient character. In another example the behaviour of a task character is changed by a body pose of the task character. In further example the location of the task in the virtual environment may be changed, e.g. by changing a parameter defining a location of the task. A task in a central region of the virtual environment, away from a wall or other boundary, may be more difficult than a task performed adjacent a wall or other boundary (where it is less likely to be observed). Such techniques can use less memory than storing each task/configuration separately, as well as facilitating task definition and reducing the processing needed for implementation.

In a related aspect there is provided a Virtual Reality (VR) therapeutic system e.g. for providing psychological therapy to a patient. The VR therapeutic system may comprise VR hardware to provide a virtual environment e.g. as previously described. Thus the VR hardware may including at least a head-mounted display unit with a sound generation capability and a VR input device. The VR therapeutic system may further comprise a computer coupled to or part of the VR hardware, e.g. a workstation.

The VR therapeutic system may be configured to implement a VR environment module to present a set of therapeutic spatial regions (scenarios) to the patient in the virtual environment using the VR system. Each therapeutic spatial region may comprise at least one patient action (scenario) task to be performed by the patient in the virtual environment by interacting with the VR system. The patient may be represented by a patient character in the virtual environment, at least part of which may be visible at least some of the time. At least one of the patient action tasks may include at least an interaction task in which the patient character interacts with a task character in the virtual environment, or a non-verbal action task in which the patient character performs a non-verbal action in relation to one or more objects in the virtual environment. At least some of the time the virtual environment may include a representation of a coach character.

The VR therapeutic system may be further configured to implement a mental anxiety measurement module to determine a mental anxiety state parameter representing a mental anxiety state of the patient by measuring, in the virtual environment, one or more characteristics of the interaction of the patient character with the task character or the one or more objects.

The VR therapeutic system may be further configured to implement a performance monitoring module to monitor performance of the patient action task in the virtual environment.

The VR therapeutic system may be further configured to implement a detector module to detect one or both of non-performance of the patient action task and a state of patient mental anxiety indicated by the mental anxiety state parameter.

The VR therapeutic system may be further configured to implement a patient feedback module to assist the patient in completion of the patient action task in the virtual environment by using the coach character to provide VR feedback comprising one or both of a verbal prompt and a visual prompt in the virtual environment in response to detection of the state of patient mental anxiety.

Further features of the VR therapeutic system may be as previously described. For example the VR hardware may include an eye tracker device and the mental anxiety measurement module may be coupled to the eye tracker device to determine the mental anxiety state parameter from the patient's gaze direction. Similarly the system may include a patient state neural network module having one or more inputs to receive the one or more characteristics of the interaction of the patient character with the task character or the one or more objects, and having an output to provide the mental anxiety state parameter representing the mental anxiety state of the patient. Further details of an example patient state neural network module are described later.

The VR therapeutic system may be partly implemented using software. For example the above described modules may be implemented in software i.e. instructions for a computer, or in hardware e.g. an ASIC (Application Specific Integrated Circuit), or in a combination of the software and hardware.

In a further related aspect there is provided a method of controlling a Virtual Reality (VR) therapeutic system for providing psychological therapy to a patient. The VR therapeutic system may comprise VR hardware to provide a virtual environment, the VR system including a head-mounted display unit e.g. with sound generation capability and a VR input device, and one or more computers coupled to or part of the VR hardware. The method may comprise presenting a set of therapeutic scenarios to the patient in the virtual environment using the VR system, wherein each therapeutic scenario comprises at least one scenario task to be performed by the patient in the virtual environment by interacting with the VR system. The patient may be represented by at least part of a patient character in the virtual environment. At least one of the scenario tasks may include at least an interaction task in which the patient character interacts with a task character in the virtual environment or a non-verbal action task in which the patient character performs a non-verbal action in relation to one or more objects in the virtual environment. At least some of the time the virtual environment may include a representation of a coach character.

The method may further comprise determining a mental anxiety state parameter representing a mental anxiety state of the patient by measuring, in the virtual environment, one or more characteristics of the interaction of the patient character with the task character or the one or more objects. The method may further comprise monitoring performance of the scenario task in the virtual environment. The method may further comprise, in response to one or both of non-performance of the scenario task and a state of patient mental anxiety indicated by the mental anxiety state parameter: assisting the patient in completion of the scenario task in the virtual environment by using the coach character to provide VR feedback comprising one or both of a verbal prompt and a visual prompt in the virtual environment e.g. in response to detection of the state of patient mental anxiety.

There is further provided one or more storage media carrying software, i.e. computer instructions, to (when running) implement the above described systems and methods.

The instructions may be configured to implement the systems and methods on one or more computers e.g. on a general purpose computer system, and/or on a mobile device, and/or on a digital signal processor (DSP) and/or on configurable or dedicated hardware. The storage media may comprise a carrier such as a disk or programmed memory e.g. a Flash drive or other memory. Software i.e. code and/or data to implement the systems and methods may comprise source, object or executable code in a conventional programming language (interpreted or compiled) such as C, or assembly code, or code for a hardware description language. Such code and/or data may be distributed between a plurality of coupled components in communication with one another.

In a further aspect there is provided a Virtual Reality (VR) therapeutic system for providing psychological therapy to a patient, the VR therapeutic system comprising a computer system (e.g. the previously described one or more computers), a head-mounted display unit for the patient, connected to or comprising the computer system; and one or more sensors for detecting patient actions performed by the patient, the sensor(s) being connected to the computer system. The computer system may be configured to cause the display unit to display a virtual environment for a patient character, the virtual environment comprising a task character in a therapeutic scenario and, temporarily or permanently, a coach character. The computer system may be further configured to cause the display unit to prompt the patient to perform a pre-defined action in relation to the task character. The computer system may be further configured to determine with the sensor(s) whether the patient has performed the pre-defined action within a first time period. The computer system may be further configured to, in response to determining that the patient has not performed the pre-defined action within the first time period, cause the display unit to have the coach character provide an additional prompt for the patient to perform the pre-defined action.

The computer system may be further configured to, after the additional prompt, determine whether the patient has performed the pre-defined action within a second time period. In response to determining that the patient has performed the pre-defined action the computer system may cause the display unit to progress the virtual environment to a different therapeutic scenario in the virtual environment or to a next level of the therapeutic scenario. In response to determining that the patient has not performed the pre-defined action within the time period the computer system may initiate an exit sequence to exit the patient from the virtual environment.

The Virtual Reality (VR) therapeutic system may further comprise a hand controller system to track the patient's hand movements e.g. one or a pair of handheld controllers and/or one or more local or remote sensors to sense a position of the hard/controller. The pre-defined action may comprise one or more of touching, holding and moving a virtual object in the virtual environment. The sensors may include a microphone and the pre-defined action may comprise speaking to the task character; the computer system may detect when the patient is speaking into the microphone in order to determine whether the patient has performed the pre-defined action.

The computer system may be further configured to obtain one or more input parameters, e.g. sensed input parameter(s), relating to behaviour of the patient, and then to adjust the first time period based on one or more of the input parameters. For example the computer system may detect when and/or how fast and/or how loudly the patient is speaking; and/or the computer system may detect a distance of the patient character from the task character in the virtual environment. In response the first time period may be shortened e.g. if the input parameter(s) relating to behaviour of the patient indicate that the patient is anxious or having difficulty performing the task, so that the coach character intervenes sooner. Ina similar way the VR system may include an eye tracking device mounted to the display unit, and an input parameter may be dependent upon time spent by the patient looking at a predefined region of the virtual environment e.g. looking downwards in the virtual environment. Also or instead one or more of the input parameters may be retrieved by the computer system from a remote data store e.g. a parameter relating to a baseline anxiety of the patient, or a parameter relating to a degree of severity of a psychological problem of the patient, or a parameter relating to the type(s) of scenario the patient finds difficult.

In implementations the computer system is configured to cause the display unit to display an initial, welcome virtual environment comprising the coach character, but not the task character, prior to displaying the therapeutic scenario virtual environment. In implementations the computer system is configured to cause the display unit to progress the virtual environment to a next level of the therapeutic scenario in response to determining that the patient has performed the pre-defined action.

In some implementations the coach character interacts with the patient character in the virtual environment by speaking to the patient character to provide the additional prompt. The VR therapeutic system may further comprise a patient mobile device with a patient sensor input to sense an anxiety level of the patient and an audio output. The mobile device is configured to process data from the patient sensor input to monitor an anxiety level of the patient when the patient is in the real world and away from the virtual environment and to provide an audio output, e.g. instructions to the patient in a voice of the coach character, in response to the detected anxiety level. The patient mobile device may comprises or consists of a wearable (or carryable) device to monitor heart rate and/or a galvanic skin response of the patient to sense the anxiety level of the patient. The audio output may be provided in response to detection of an increased anxiety level, e.g. above a threshold, and/or increasing by more than a threshold, and/or having a pattern indicating patient anxiety.

Some implementations of the VR therapeutic system collect patient data while the patient is in the therapeutic scenario in the virtual environment. This data may comprise the mental anxiety state parameter representing the mental anxiety state of the patient and/or the one or more measurements or characteristics from which this is derived. For example the patient data may comprise the previously described measurement(s) such as one or more of: proximity measurements, eye gaze direction measurements, patient verbalization response time measurements, task performance/non-performance measurements, task duration (elapsed time) measurements, and real-world biometric measurements. Also or instead the patient data may comprise processed versions of such data, for example a difference between a biometric anxiety measurement, such as cortisol level, heart rate or GSR, at the start and end of a scenario. The patient data may be stored locally in the one or more computers (computer system) and/or stored remotely e.g. in a remote server. In general connections between components of the VR therapeutic system may comprise wired and/or wireless connections; data may be stored during or after completion of a VR session or of a particular scenario task in the session.

As previously mentioned, some implementations of the VR therapeutic system include a patient (mobile) device; such implementations may also include the remote server. The remote server may be configured to receive the patient data from the computer system collected while the patient is in the therapeutic scenario in the virtual environment, the patient data representing e.g. performance of the patient in the virtual environment therapeutic scenario. The patient device may be configured to communicate with the remote server and to output data to the patient dependent upon the performance of the patient in the virtual environment therapeutic scenario e.g. to facilitate performance of the patient in a real world scenario corresponding to the virtual environment therapeutic scenario.

The patient device may be configured to collect further patient data, for example corresponding to that described above, dependent upon the performance of the patient in the real world scenario corresponding to the virtual environment therapeutic scenario, and to transmit this to the computer system via the remote server. The remote server may be configured to adjust the virtual environment therapeutic scenario dependent upon the further patient data such that the virtual environment therapeutic scenario is adapted dependent upon the performance of the patient in the real world scenario.

Some implementations of the VR therapeutic system comprise memory storing data for displaying a plurality of different virtual environments for plurality of different therapeutic scenarios. The or other memory may also store a patient model associated with the patient, the patient model comprising one or a plurality of model parameters. The computer system may be configured to map the patient model associated with the patient to a subgroup of the therapeutic scenarios based on the model parameters for presentation of the subgroup of therapeutic scenarios to the patient. For example the patient model may be dependent on the previously described patient data, such as the cortisol level, and this may be used to select one or more of the therapeutic scenarios/tasks for the patient.

The computer system may also be configured to cause the head-mounted display unit to present to the patient the subgroup of therapeutic scenarios to enable the patient to choose a therapeutic scenario. Each of the subgroup of therapeutic scenarios may have a plurality of difficulty levels, and the computer system may be further configured to map the patient model to a difficulty level dependent upon the model parameters. The computer system may be further configured to receive as input from the one or more sensors, information identifying patient actions in the virtual environment, and to update the model parameters of the patient model based on the input.

Some implementations of the VR therapeutic system are configured to use the collected (and stored) patient data to predict a therapeutic scenario and/or difficulty level for a patient. This may be the patient from whom the patient data was collected, or another patient.

For example in one implementation the patient data relating to a therapeutic scenario is processed to determine a progression or outcome measure for the patient, for example from a difference between measurements such as cortisol, heart rate and the like before and after the therapeutic scenario. The progression or outcome measure may then be used to select another therapeutic scenario or another difficulty level for the same therapeutic scenario for presentation to the patient. Alternatively a subgroup of such therapeutic scenarios or difficulty levels may be selected and presented to the patient, to enable the patient to select a next scenario/level.

Thus in some implementations the VR therapeutic system (and corresponding method) may include a cortisol sensor to sense a level of cortisol in the patient. The system/method may then determine a measure of performance of the scenario task or pre-defined action, e.g. an outcome measure, dependent upon the level of cortisol in the patient. Also or instead the system/method may determine a scenario task (e.g. a type and/or level of therapeutic scenario), or a pre-defined action for presentation in the virtual environment dependent upon the sensed level of cortisol in the patient.

In the same or another implementation a machine learning classifier such as a feed forward or recurrent neural network classifier may be employed to process the patient data for a patient to determine classification output comprising comprise data identifying one of a set of classes. Each class may correspond to a therapeutic scenario and/or difficulty level for the patient e.g. most likely to be beneficial to the patient. Also or instead the classification output may be used to determine a set of therapeutic scenarios and/or difficulty levels for the patient. Also or instead the classification output may be used to predict a number of therapeutic scenarios needed by the patient until a particular value of the progression or outcome measure is achieved e.g. representing a successful final outcome. A prediction may be made for a new patient e.g. by presenting the patient with a test scenario. In some implementations classification output may comprise a set of probability values or scores, one for each class e.g. therapeutic scenario or set of therapeutic scenarios. In some implementations the machine learning classifier may comprise a feed-forward classifier such as a deep neural network e.g. multilayer perceptron (for which time-dependent patient data may be aggregated over time), or a recurrent neural network i.e. a neural network with one or more recurrent neural network layers. The machine learning classifier, e.g. neural network, may be trained using a supervised learning technique based on historical patient data and/or psychological measures of historical patient outcomes established using existing techniques.

Thus in some implementations the VR therapeutic system (and corresponding method) may be configured to collect patient data characterizes performance of the patient on a scenario task or pre-defined action. The system/method may then use a machine learning subsystem configured to process the patient data to determine a type or level of therapeutic scenario (or a set of these), a number of therapeutic scenarios or levels of therapeutic scenario for presentation to the patient in the virtual environment, e.g. a number estimated to be required for a successful final outcome.

Thus in a further aspect there is provided a Virtual Reality (VR) therapeutic system for providing psychological therapy to a patient. The VR therapeutic system may comprise a VR system to provide a virtual environment, the VR system including at least a head-mounted display unit with a sound generation capability and a VR input device. The VR therapeutic system may further comprise one or more computers coupled to or part of the VR system. The computer(s) may be configured to present a set of therapeutic scenarios to the patient in the virtual environment using the VR system, wherein each therapeutic scenario comprises at least one scenario task to be performed by the patient in the virtual environment by interacting with the VR system. The patient may be represented by a patient character in the virtual environment. At least one of the scenario tasks may include at least an interaction task in which the patient character interacts with a task character in the virtual environment or a non-verbal action task in which the patient character performs a non-verbal action in relation to one or more objects in the virtual environment. At least some of the time the virtual environment may include a representation of a coach character.

The computer(s) may be further configured to implement a patient state neural network having a neural network input to receive: i) one or more characteristics of the interaction of the patient character with the task character or the one or more objects in the virtual environment or ii) sensor data to make an anxiety measurement, sensing e.g. one or more of a heart rate, galvanic skin response, and cortisol level of the patient in the real world. The patient state neural network may process the neural network input to determine a mental anxiety state parameter representing a mental anxiety state of the patient.

The computer(s) may be further configured to monitor performance of the scenario task in the virtual environment.

The computer(s) may be further configured to, in response to one or both of non-performance of the scenario task and a state of patient mental anxiety indicated by the mental anxiety state parameter, assist the patient in completion of the scenario task in the virtual environment. For example the VR system may assist the patient in completion of the scenario task by using the coach character to provide VR feedback comprising one or both of a verbal prompt and a visual prompt in the virtual environment e.g. as previously described. Also or instead the VR system may assist the patient in completion of the scenario task by modifying the therapeutic scenario to change a difficulty level of the therapeutic scenario, e.g. online (i.e. during the selected scenario), or by halting the scenario and starting a new scenario/task. The therapeutic scenario may be modified online to reduce a difficulty of the scenario as previously described e.g. by reducing a level of background noise, or a number of task characters in the scenario, and so forth.

In implementations because the patient state neural network (e.g. classification) output determines the mental anxiety state parameter which is used to control the coach character and/or scenario difficulty, the patient state neural network forms part of a closed-loop feedback (to the patient) system. Thus the mental anxiety state parameter may be used to adjust or control the experience of the patient in the VR environment, e.g. to calm the patient e.g. by spoken output, when patient anxiety is detected.

In implementations the patient state neural network may be as previously described. For example the patient state neural network may comprises one or more initial input neural network layers configured to receive the neural network input and to generate a latent representation of the neural network input. The patient state neural network may further comprise at least one intermediate neural network layer having an intermediate layer input coupled to the one or more input neural network layers, and having an intermediate layer output. The patient state neural network may further comprise one or more output layers e.g. having an input coupled to the intermediate layer output to generate the neural network output. The neural network output may define a score distribution over a discrete set of possible patient anxiety values. The patient state neural network may be configured to generate the mental anxiety state parameter from the score distribution. The mental anxiety state parameter may be a categorical variable having two or more anxiety values e.g. defining an anxious state and a not-anxious state (or having more anxiety gradations). The patient state neural network may then select a value, e.g. a most likely anxiety value, according to the score distribution.

In a further aspect the therapeutic VR system may comprise a subsystem, which may be termed an adaptive bio-behavioural system, configured to adapt the course of treatment of the patient based on the patient's behaviour and/or biological response while in the virtual environment.

In a virtual scenario the scenario progresses based on a scenario specific algorithm also referred to as a decision tree. The virtual scenario progresses down different branches of the decision tree based on the patients actions in the virtual environment. For example, by completing a scenario task the virtual scenario progresses along a first branch of the decision tree, whereas non-completion of said task causes the virtual scenario to progress along a second branch of the decision tree.

The adaptive bio-behavioural system may be configured to change the decision tree/algorithm used to advance the patient through a particular virtual scenario. That is, instead of only using such measurements to correctly follow a pre-set decision tree with a limited number of options, the adaptive bio-behavioural system can adapt the decision tree itself (e.g. by including further options or introducing new end points). The adapted tree may then be used as a pre-set tree in the specific virtual scenario in subsequent therapeutic sessions. The adapted decision tree/algorithm may be used for future VR therapeutic systems and coach's functions.

A specific virtual scenario can be configured to provide different stimulus intensities (e.g. by the number of virtual characters, the number of interactions with the virtual characters, and by the type of interaction). The adaptive bio-behavioural system may be configured to adapt the decision tree based on the stimulus intensity.

In one implementation, the adaptive bio-behavioural system uses implicit metrics (e.g. reaction time and/or a measure of conformity to an expected response to stimulus) measured while the patient is in the virtual environment to adapt the course of the treatment program provided to the patient by the therapeutic VR system within and/or outside the VR environment.

Web and/or mobile data sources (e.g. user input on a smartphone between VR sessions) are used to integrate with the therapeutic VR system to adapt the level of a therapeutic scenario or stimulus intensity.

The system may be used in combination with other programs, interventions (e.g. behavioural and/or pharmacological), and other digital therapeutic technologies.

The patient's performance (or non-performance) of tasks within VR environment may also be used to assess the patient's understanding of therapeutic material (e.g. psychoeducation), which can be used to trigger further VR content based on the determined level of understanding.

In one implementation, position and/or body tracking during scenario tasks and during event-specific stimulus is used as input to the adaptive bio-behavioural system. The inputs can be used in an algorithm to adapt to the patient's level of engagement and performance during the task or stimulus.

### BRIEF DESCRIPTION OF THE DRAWINGS

These and other aspects of the system are now described with reference to the following drawings.

FIG. 1 shows a schematic diagram of a VR therapeutic system according to an embodiment;

FIG. 2 shows a flow diagram of a method performed by a VR system according to an embodiment;

FIG. 3 shows a schematic diagram of a VR therapeutic system according to another embodiment;

FIG. 4a shows a flow diagram of a method performed by a VR system according to an embodiment;

FIG. 4b shows a flow diagram of a method performed by a VR system to determine a mental anxiety state parameter according to an embodiment;

FIG. 5 shows a schematic diagram of a system for providing therapeutic treatment using a VR system and a mobile device according to an embodiment;

FIG. 6 shows a flow diagram of a method performed by a VR therapeutic system according to an embodiment providing a scenario with a bus;

FIG. 7 shows a flow diagram of a method performed by a VR therapeutic system according to an embodiment providing a scenario in a cafe;

FIG. 8 shows a flow diagram of a method performed by a VR therapeutic system according to an embodiment providing a scenario in a shop;

FIG. 9 shows a flow diagram of a method performed by a VR therapeutic system according to an embodiment in order to position the patient correctly before changing scenario or level;

FIG. 10 shows a flow diagram of a method performed by a VR therapeutic system according to an embodiment providing a virtual welcome room;

FIG. 11 shows a flow diagram of a method performed by a VR therapeutic system according to an embodiment wherein an anxiety rating of the patient is determined;

FIG. 12 shows a flow diagram of a method performed by a VR therapeutic system according to another embodiment wherein the anxiety rating of the patient is determined; and

FIG. 13 is a schematic diagram of a clinical platform comprising a VR platform for treating multiple mental health conditions.

In the figures like elements are indicated by like reference numerals.

### DETAILED DESCRIPTION

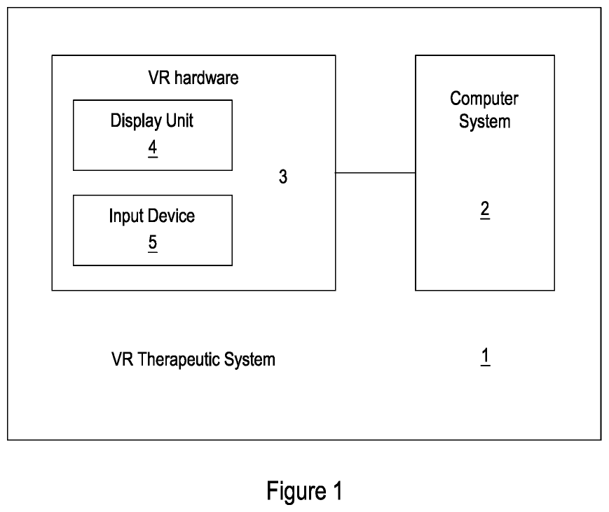

FIG. 1 shows a schematic diagram of an example of a Virtual Reality (VR) therapeutic system 1 (simply referred to as ‘VR system’ herein) comprising a computer system 2, and VR hardware 3 comprising a wearable display unit 4 for the patient, and an input device 5.

The computer system is connected to the wearable display unit and configured to cause the display unit to display a virtual environment for a patient character. The computer system may be a standalone workstation, a computer network, or may be a small computer that is integrated into the wearable display unit or otherwise carried by the patient.

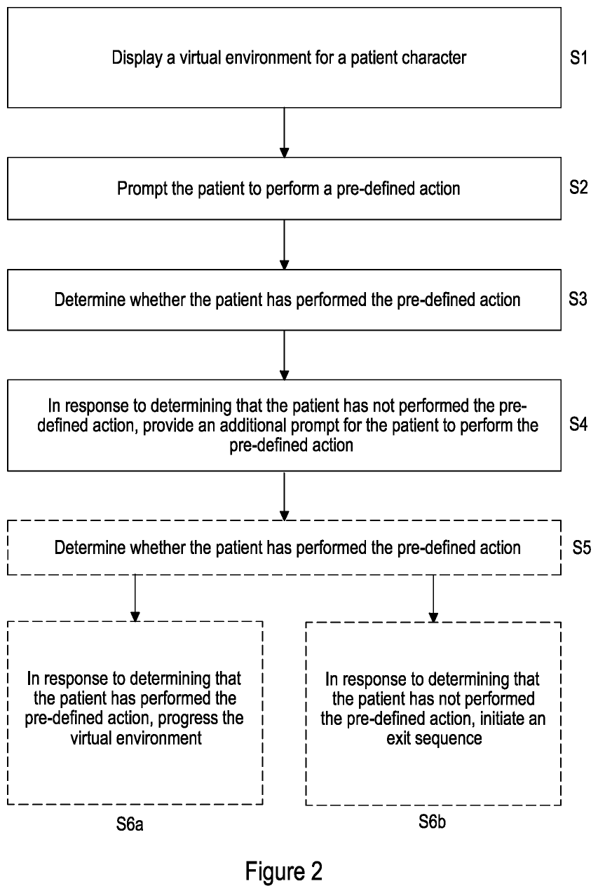

FIG. 2 is a flow diagram illustrating the steps of a method of providing a therapeutic session using a VR system comprising a computer. The computer is configured to display a virtual environment for a patient character (step S1), prompt the patient to perform a pre-defined action (step S2), determine whether the patient has performed the pre-defined action (step S3), and in response to determining that the patient has not performed the pre-defined action, provide an additional prompt for the patient to performed the pre-defined action (step S4). Optionally the computer is further configured to, after providing the additional prompt, determine whether the patient has performed the pre-defined action (step S5), and in response to determining that the patient has performed the pre-defined action, progress the virtual environment (step S6a), or in response to determining that the patient has not performed the pre-defined action, initiate an exit sequence (S6b).

To implement VR therapy sessions using the VR system 1, the system uses 3D environments and programming of interactions. The software used to develop the VR sessions can be classified in two main categories a) 3D modelling and animation software and b) development engines. Within category a) (3D modelling and animation software) three-dimensional objects are generated by using the techniques of CAD software, as well as the visual aspect of surfaces, the illumination of the surroundings, effects of nature, dynamic effects (forces, gravity, etc.), and all kinds of animation. This type of software is also used in the production of films, videogames, and projects. 3D modelling software that may be used includes, Blender™, Autodesk 3DStudio Max™, and Autodesk Maya™. Regarding the second category b), different names are used to describe it, including development engines, videogame engines, and graphic engines (they generate the interactive images used in videogames or VR applications). The word “engine” refers to a program, or a part of it, that executes a certain type of task. Development engines provide a set of basic programmed functions that are common in all VR applications: a) a rendering engine to generate 2D and 3D graphics; b) a collision-detecting engine; c) possible interactions with the environment; d) sounds and music; e) animation; f) artificial intelligence; g) communication with the network; h) memory management, etc. Examples of graphic engines that may be used include Unity3D™ and Unreal Engine™.

The development of VR sessions can also be divided into three categories a) Environment, b) Characters and c) Scripting:

a) Environment. This encompasses the passive elements of the scenario, all that is displayed that will remains static.
b1) Character creation. Creating the characters, and all the logics underneath to puppeteer them.
b2) Character animation. This is the process to animate the characters created in (b1) and make them move. Character animators can use Autodesk Maya and Autodesk MotionBuilder. The animations can be created with a motion capture system such as Optittrack.
c) Scripting. Using a game engine (e.g. Unity) all the elements are put together, to set the locations and program the logics and behaviour.

Each scenario will have the same environment model. The Cafe will be the same cafe for all levels 1 to 5. The characters on each level may change and some characters will be in certain levels and not others.

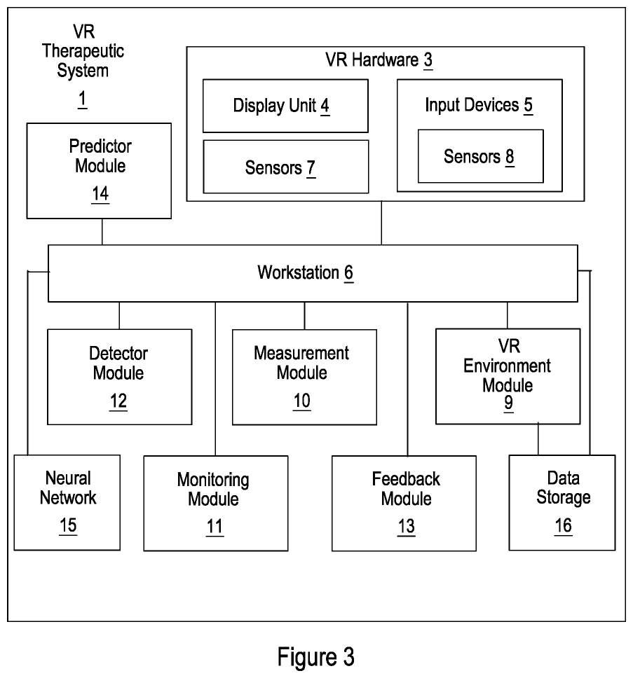

FIG. 3 shows a schematic diagram of an example of a VR therapeutic system 1 comprising a workstation 6, VR hardware 3 comprising a display unit 4 with audio capabilities, on or more input devices 5, and a plurality of sensors 7 and 8. One of the sensors 7 in the display unit is an eye tracking sensor for measuring the gaze direction of the patient character in the virtual environment. Another sensor 7 in the display unit is a position tracking sensor for tracking the patient's position. Other sensors 8 outside the display unit include tracking and input sensors mounted to a handheld controller (one of the input devices 5) for tracking the patient's hand movements in the virtual environment. A biosensor on the handheld controller can be used to measure sweat/cortisol levels, which can be used to evaluate the patient's anxiety level during a therapeutic session. The sensors 7 and 8 also includes a microphone for speech detection. The VR system 1 further comprises a plurality of modules including a VR environment module 9, a measurement module 10, a monitoring module 11, a detector module 12, a feedback module 13, a predictor module 14 and a patient state neural network module 15. The VR system is configured to implement the VR environment module 9 to present a set of therapeutic spatial regions to the patient in the virtual environment using the VR system 1. These spatial regions correspond to different therapeutic scenarios. The VR system 1 is further configured to implement the measurement module 10 to determine (together with the neural network module 15) a mental anxiety state parameter representing a mental anxiety state of the patient, to implement the monitoring module 11 to monitor performance of a patient action task, the detector module 12 to detect non-performance of the task and/or the state of mental anxiety of the patient, and the feedback module 13 to assist the patient in completion of the task. The VR system 1 further comprises a data storage 16, for storing data relating to patients and to the virtual environment, which is accessible by the VR environment module 9 and the workstation 6 in order to render the virtual environment. The VR system 1 is configured to implement the predictor module 14 to predict how many sessions or which spatial region (scenario) and difficulty level, a patient is recommended to pursue. The predictor module 14 preferably uses machine learning to provide predictions. The workstation 6, modules (9 to 15), and data storage 16 may form the computer system 2 of the VR system in FIG. 1.

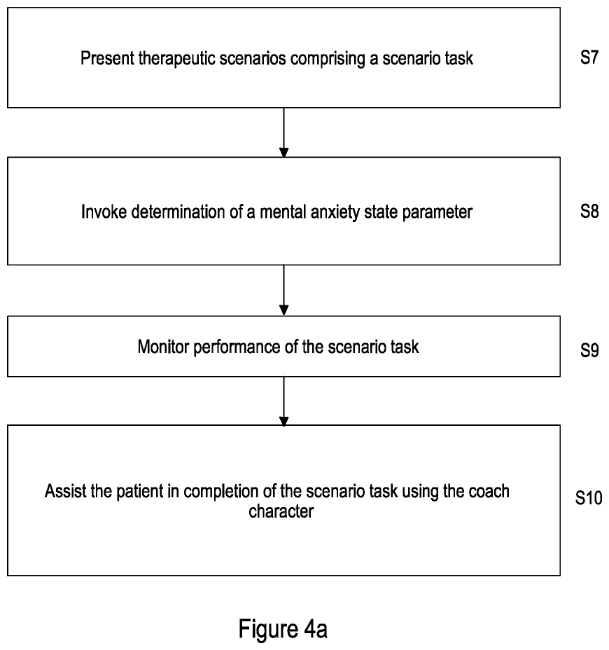

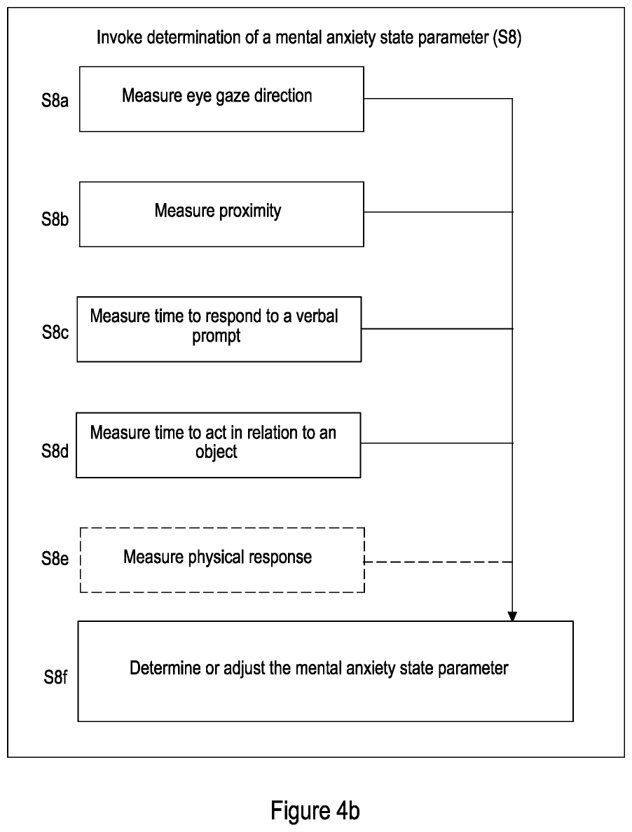

FIG. 4a is a flow diagram illustrating the steps of a method of providing a therapeutic session via VR system such as the VR system illustrated in FIG. 1 or 3. The method comprises presenting therapeutic scenarios comprising a scenario task (step S7), invoke determination of a mental anxiety state parameter (step S8), monitoring performance of the scenario task (step S9) and assisting the patient in completion of the scenario task using a coach character (step S10). The mental anxiety state parameter can be determined from a range of different parameters, relating to the patient character's interaction with the virtual environment.

FIG. 4b is a flow diagram illustrating an example of the VR system invoking the determination of the mental anxiety state parameter (step S8). VR system can invoke the measurement module 10, to measure eye gaze direction of the patient character (step S8a), proximity of the patient character to objects or other characters in the virtual environment (step S8b), time for the patient character to respond to a verbal prompt (step S8c), time for the patient character to respond in relation to an object in the virtual environment (step S8d) and optionally a physical/biological response of the patient (e.g. cortisol level) (step S8e). One or more of these measurements are used to determine or adjust the mental anxiety state parameter of the patient (step S8f), which in turn is used by the feedback module 13 to adjust the assistance provided to the patient character. In general, if the state parameter indicates a high level of anxiety, then the feedback module will be implemented to provide a prompt or encouragement from the coach character more quickly. Any one of the measurements can be invoked in response to an event in the scenario (e.g. movement of other characters or objects in the environment), or at given time intervals (e.g. every second). The proximity measurements may be triggered by movement of the patient character or by movement of an object or other character in the virtual environment. Eye tracking may be triggered by the patient character moving in front of a task character in a scenario, and can be used to determine if the patient character keeps eye contact with the task character.

In an example, the neural network module 15 is used to determine the mental anxiety state parameter. Inputs to the neural network module 15 comprises the one or more measurements in the virtual environment (steps S8a to step S8d) from the measurement module 10 and/or the physical measurements (step S8e) from sensors 8. The neural network 15 may directly output the mental anxiety state parameter, or a set of scores or probability values, one for each of a set of classification categories, which may represent a degree of mental anxiety of the patient. These scores can then be used to classify the patient as anxious or not anxious, and/or to grade patient anxiety into more than two categories.

The VR system 1 makes use of techniques from Cognitive-Behavioural Therapy (CBT), an evidence-based treatment for a number of mental health conditions. It is an interactive treatment, which allows patients to test their automatic beliefs and feelings in virtual environments in order to change their reaction to those automatic responses.

In an example, the VR system 1 has access to a plurality of different therapeutic scenarios, which it can present to the patient in the virtual environment. The VR system is configured to provide the patient several levels, carefully graded in difficulty and to test the patient's response and behaviour. The predictor module 14 can be used to select a subgroup of scenarios to provide to a specific patient. For example to treat social avoidance, the system can use different scenarios comprising a café, a shop, a pub, a doctor waiting room, a bus and a street. Each scenario is associated with a task/action to be completed by the patient's virtual character and a difficulty level, which determines certain aspects of the scenario.

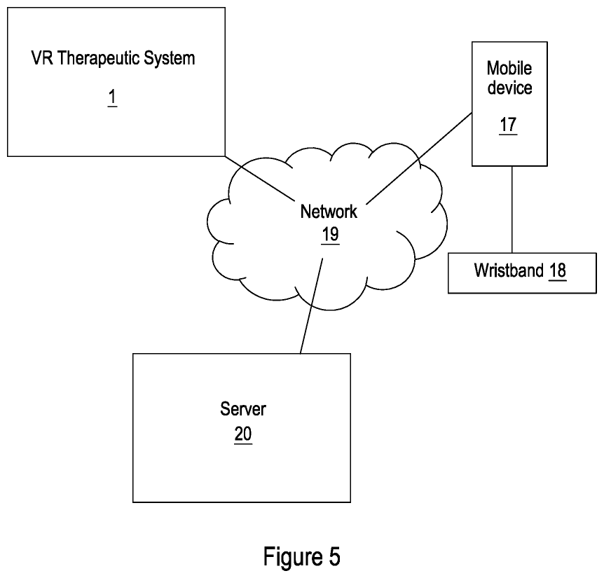

FIG. 5 shows a schematic diagram of a system comprising the VR therapeutic system 1 and a mobile device 17 with a mobile application for providing further treatment and to gather patient data in between VR sessions. The mobile device 17 is connected to a wristband 18, which can be worn by the patient and which comprises sensors for gathering patient data (e.g. heart rate, GSR, cortisol/sweat level) that can be transmitted to the mobile application. The mobile device 17 and VR system 1 can communicate over a network 19 directly or via a server 20 (e.g. a cloud-based server) to share data. The mobile application can use the patient data from the wristband 18 to determine an anxiety rating of the patient, and to provide feedback based on the anxiety rating. For example, if the mobile application determines that the patient has a high anxiety level, then the mobile application may provide verbal encouragement from the mobile device. Preferably, the voice giving verbal encouragement is the same voice as that of the coach character in the virtual environment. The mobile device 17 or wristband 18 can track the patient's location using GPS tracking and provide the location to the application. The application can use the location to provide targeted feedback to the patient. For example, the mobile application may use the location to identify when the patient is in a real-world scenario corresponding to one of the therapeutic scenarios in the virtual environment and provide appropriate feedback for that situation.

The patient mobile device may thus use the monitored patient activity/anxiety to identify when the patient is in a stressful real world situation. In response, the mobile device 17 may then provide real world feedback e.g. a spoken audio output to the patient via headphones in the voice the coach character uses in the virtual environment to assist the patient. The voice may provide calming words and/or spoken instructions or encouragement to the patient from the mobile device 17.

In an example, the mobile application collects information and feedback provided by the patient and sends patient data to the VR system 1. The VR system 1 uses the patient data provided by the mobile application to modify the VR therapy session for that patient. Similarly, patient data collected by the computer system during a VR session (e.g. patient progress, level achieved, and learnings) can be sent from the VR system to the mobile application. Between VR sessions, the mobile application can reinforce learning points and encourages patients to practice skills and behaviours learnt in the VR session, using the information provided by the VR system 1.

In an example, an automated CBT program is provided to the patient from the VR system 1. Time spent in scenarios, answer to questions, level achieved and goals are collected while the user receives the VR treatment. The patient data may be stored locally in the computer system of the VR system 1 and/or stored remotely in the remote server 20. The data is passed to the server 20 or directly to the mobile device 17 for processing. In general, connections between components of the VR therapeutic system may comprise wired and/or wireless connections; and the data may be stored during or after completion of a scenario task.

Between sessions, the mobile application uses this data to reinforce learning points and encourages user to apply the learnings in the real world. The mobile application provides an interface for the patient to give feedback, and record if he has managed to apply learning points in real situation and provide a rating. This feedback can be sent back to the server 20 or directly to the VR system 1. In the next VR session, the automated treatment is adjusted using the processed feedback to tailor treatment to the user need.

Each scenario may have five difficulty levels, within which the VR system is configured to:

### Café Scenario

1. Provide a queue and prompt the patient to order a drink.
2. Provide more background characters (busier cafe). Provide a queue and prompt the patient to order a drink.
3. Provide a queue and prompt the patient to order a drink. Provide a barista character that asks the patient character to repeat the order.
4. Provide a customer character that leaves his wallet on the table and prompt the patient to alert the customer character.
5. Provide a child character that blows bubbles into the air and prompt the patient to pop the bubbles. Provide further background characters that watch the patient character when performing the task.

### Bus Scenario

1. Prompt the patient to wait at a bus stop, and then to pay and step into the bus.
2. Provide more background characters (busier). Prompt the patient to wait at a bus stop, and then to pay and step into the bus.
3. Provide more background characters (busier). Prompt the patient to wait at a bus stop, and then to pay and step into the bus.
4. Provide a passenger character that asks the patient character to ring the bell while other passenger characters look at the patient character.
5. Provide further background characters (very busy). Prompt the patient to wait at a bus stop, and then to pay and step into the bus.

### Street Scenario

1. Prompt the patient to leave the front door and wait for a taxi on a street
2. Provide more background characters (busier). Prompt the patient to leave the front door and wait for a taxi on a street
3. Provide more background characters (busier). Prompt the patient to leave the front door and wait for a taxi on a street
4. Prompt the patient to leave the front door and to wash graffiti off a wall with his back turned to the street
5. Provide more background characters (very busy). Prompt the patient to leave the front door and wait for taxi on a street

### Doctors Waiting Room (GP) Scenario

1. Prompt the patient to wait in a queue.
2. Prompt the patient to wait and to give personal information to a receptionist.
3. Provide virtual leaflets that blow into the air and prompt the patient to catch them while other characters watch the patient character.
4. Provide a slightly angry person character that asks the patient character to pass a pot of pens.
5. Prompt the patient to wait to be called by a doctor, and to ask a receptionist when it is his turn.

### Pub Scenario

1. Prompt the patient to wait in a pub for a friend character to arrive.
2. Provide more background characters (busier). Prompt the patient to wait in a pub for a friend character to arrive.
3. Provide more background characters including loud sports fans (busier). Prompt the patient to wait in a pub for a friend character to arrive.
4. Provide a sports fan character that asks the patient character where the toilets are.
5. Provide a barman character that asks the patient character to ring the last orders bell while other characters look at the patient character.

### Shop Scenario

1. Prompt the patient to browse shop while a shopkeeper character looks at the patient character
2. Provide other customer characters in the shop
3. Prompt the patient to pick up three items from a shelf with his back turned to the shop
4. Provide more background characters (busier). Prompt the patient to pick up three items from a shelf with his back turned to the shop
5. Provide further background characters (very busy). Prompt the patient to wait at the till while the shopkeeper character rings up the patient character's items.

The difficulty may be further adjusted by adjusting the length of time that the user is given on a given level of a scenario. The difficulty may also be adjusted by changing the behaviour of the background characters, for example by adjusting the frequency and/or duration at which they look at the patient character.

Whenever an action is required by the patient, the VR system determines whether the patient has successfully completed the action within a given time period and responds accordingly. The prompts occur in the context of the scenario and may be delivered verbally by one of the characters in the situation and/or by a coach character. They may also involve drawing the patient's attention to specific objects that are required to complete the level. The system sets a fixed number of prompts (typically at least two) before the system determines that the patient will not complete the task, and in response to the determination causes the scenario to end. If a patient does not successfully complete a level, then the system will automatically (after a time period from the last prompt) exit the patient from that scenario, so that the patient does not stay in the level indefinitely. To exit the patient character from the scenario the system triggers an “outro” sequence, and the patient is not offered the opportunity to proceed: they must repeat the level in order to progress. This is due to the careful gradation of difficulty in the program to maximize learning and ensure therapeutic benefit. This interaction is critical for the key concept of ‘presence’ in VR environments, which relates to the feeling that the patient is ‘really there’ and believes and behaves as if they were a part of the virtual world represented. When characters, environments, and the structure of the program do not behave credibly, the patient may have a break in presence, which limits the effectiveness of VR treatment.

Difficult situations are coached through and, when progress is made, patients can progress. The pacing of the treatment is key for its effectiveness and the prompts ensure that only patients who are ready to proceed by successfully completing the level do. Because the interaction is done by the characters in situ as well as the Coach, it mirrors reality by placing the patients in situations that they will actually be in, therefore ensuring the therapeutic benefit is especially relevant and therefore produce better outcomes. The interaction also creates a degree of rapport between the patient and the coach character, who is relied upon for prompts whenever present. This is helpful as the patient can expect to have encouragement in testing out their beliefs in especially difficult situations within the VR and know they will be prompted if they do not immediately complete the action.

Known VR CBT products have no interactions in the virtual world. This means that presence is limited as there will often be a practitioner or clinician in the room with the patient who is speaking as the patient is in the virtual environment. Many programs do not feature any activities, but merely involve exposure to frightening situations. There is no ability to test out new responses to automatic thoughts and feelings.

The VR system can provide different objects in the virtual environment with which the patient can interact using the hand controller input device or the microphone to make selections and to complete tasks:
Orb Selector. The patient character can reach and grab an orb relating to their choice (to select a scenario, the orb is displayed underneath a floating 2D image representing of the scenario). When grabbed, the orb will burst or otherwise give an indication confirming the choice. The orb selector can allow the patient to select what scenario they want to do, what level to start on, whether they feel safer (yes/no), or if they are ready with a single selection.
Orb Slider. The Coach can ask the patient how confident they feel going into different situations. The patient can reach and grab an orb in front of them and move it on a number line (e.g. marked 1-10) to indicate their confidence level. Such indications are logged throughout the course of the session.
Pen Pot Sliding. The patient character can reach and slide the pot of pens towards another character in the Doctor's waiting room in level 4.
Glowing Footprints. The patient may see glowing footprints when prompted to move to the correct location.
Glowing Object Prompts. Certain objects can glow if a patient needs to interact with the.
Carryable Objects. Carryable objects are picked up by squeezing the trigger on the controller when the patient's hand is near the object and dropped by releasing the trigger. Certain objects like the hose (Street Level 4) are not dropped when the trigger is released, but can still be passed from hand to hand. Certain objects like the Payment Card (in the Bus and Shop scenarios) are attached to the patient's hand upon the start of the level. If the object has a trigger action (like the Hose water flow) it is controlled by how much the patient is squeezing the trigger.
Voice Detection. Patients will be required to speak—it may not be necessary to implement voice recognition, and instead only detect whether a patient has spoken or not.

An example of a physical non-verbal interaction is provided in the bus scenario, where the patient must pay each time when entering the bus. If they do not, the coach (if present) will prompt the patient to pay. The payment machine will also visually glow to draw the patient's attention and indicate the required action of paying.

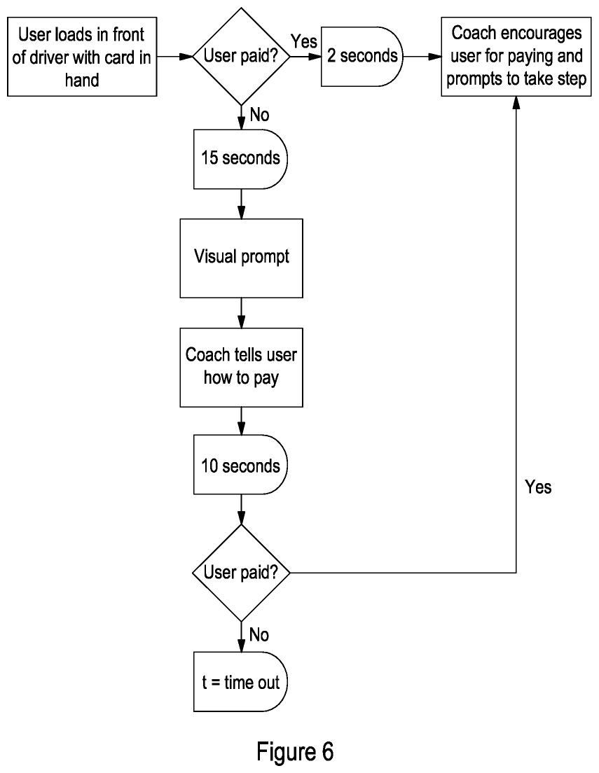

FIG. 6 is a flow diagram illustrating some of the steps performed by the VR system in a therapy session comprising the bus scenario. The patient character loads in front of the bus driver with a virtual payment card in his hand. The VR system determines if the patient pays the driver based on input from the hand controller, which tracks the motion of the hand that holds the virtual payment card. If the system determines that the patient character has paid, then the coach provides positive feedback (encouragement) to the patient character and prompts him to take a step. If the system determines that no payment has been made within a time period of 15 seconds, then the system provides a visual prompt followed by verbal prompt through the coach character by telling the patient character how to pay. The system determines if the patient character performs the required task within a time period of 10 seconds, and if not exits the scenario.

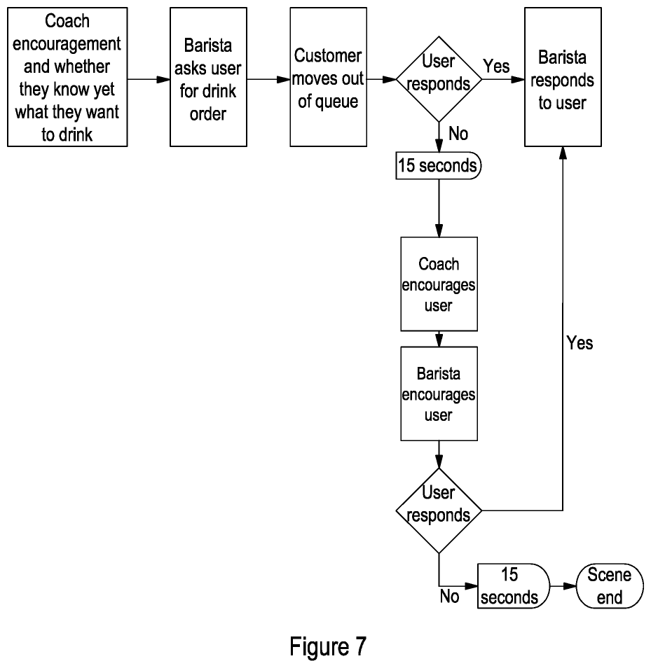

FIG. 7 is a flow diagram illustrating some of the steps performed by the VR system when running the café scenario. After the patient character has waited in the queue for his turn, the coach gives verbal encouragement to the patient and asks if he knows what he wants to drink. The barista character asks the patient character to order a drink. The patient character is thereby prompted to step forward out of the queue and order a drink from the barista character. Position tracking and voice recognition are used to determine if the patient completes the task. In response to completing the task, the barista character responds to the patient character and the patient can proceed to the next level. If the patient character does not step forward and no speech is detected within a time period of 15 seconds, then the VR system determines that the patient has not performed the task. In response to the determination of non-performance, the coach provides encouragement to the patient character and the barista provides an additional prompt to order. Again, in response to determining that the patient has performed the task, the VR system progresses the barista responds to the patient character and the patient can progress. If the patient does not order within 15 seconds of the second prompt from the barista, the system determines that the patient has not performed the task and ends the scenario.

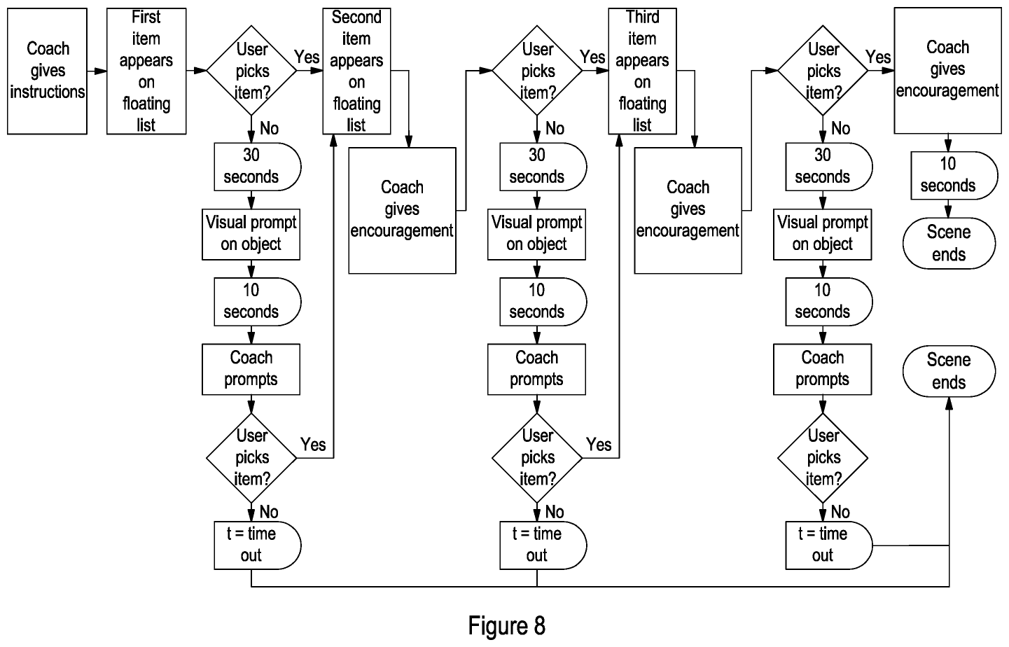

FIG. 8 is a flow diagram illustrating some of the steps performed by the VR system in a therapy session comprising the shop scenario, where the pre-defined action that the patient is prompted to perform comprises selecting three items from a list and placing them in a basket in the shop. The coach provides instructions to the patient and a first item on a floating list appears. The VR system determines if the patient character picks the item through input from the hand controller. In response to the patient character picking the item, the VR system provides the second item on the list and the process is repeated. In response to the VR system determining that the patient has not performed the task within a time period of 30 seconds, the system provides a visual prompt related to the item, and after a further 10 seconds provides a prompt through the coach. If the patient performs the task in response to these prompts, then the VR system progresses the scenario to the next item. If the VR system determines that the task has not been completed after providing the additional prompts, then the scenario is ended.

When moving between scenarios or between different levels of a scenario, the patient will have to reposition himself to the starting point, which could be different from the previous starting point. The VR system is configured to prompt the patient to move to the correct position and to determine when the patient character is in the correct position.

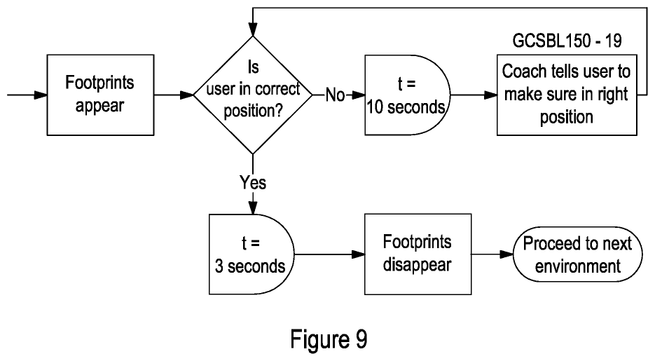

FIG. 9 is a flow diagram illustrating the steps performed by the VR system to reposition the patient before starting a new scenario or a new level. The VR system provides footprints on the floor/ground of the virtual environment to indicate the correct position. The system determines if the patient character is standing on the footprints by tracking the patient's position relative to the virtual environment. If the patient is not in the correct position after a time period of ten seconds, then the coach provides a verbal prompt to move to the right position. In response to determining that the patient character is standing on the footprints, the footprints are removed and the VR system proceeds to display the next level or scenario.

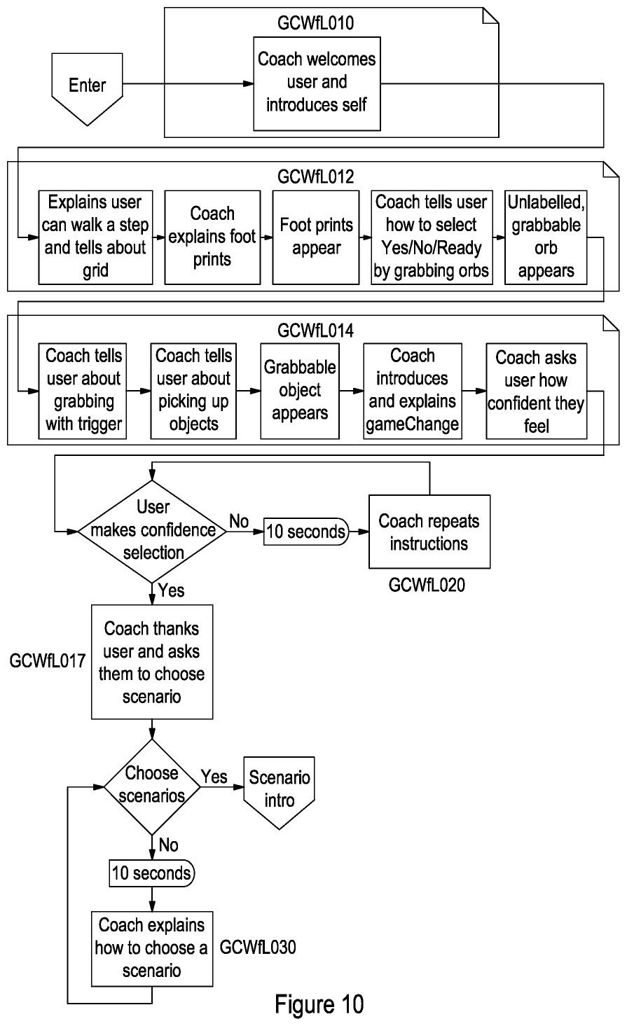

Before starting a specific scenario (bus, café, shop etc.) the VR system can provide a virtual environment comprising a “welcome room”, where the coach can be introduced to the patient and where, in some examples, the patient can select a scenario for the therapy session. FIG. 10 is a flow diagram illustrating the steps associated with the welcome room performed by the VR system. The patient character enters the welcome room. The coach explains how the patient character can interact with the virtual environment. Virtual objects with which the patient character can interact (e.g. an orb selector) are provided. The coach asks the patient to indicate a confidence level before he selects a scenario for the therapy session.

The interaction of the coach within the virtual environment is provided in response to the system determining whether the patient successfully completes an action. The determination can comprise measuring patient input associated with the specific action required (verbal, physical, or both) within a specified time period. The specific time period for a given task can be based on careful study of what is appropriate and expected for a given action, which leads to verbal and visual prompts when the patient is unsuccessful to enable the patient to complete the given task and move on to the next level/scenario.

The time period can preferably be determined or adjusted in-situ. For example, in one example of the VR system, wherein the display device comprises eye-tracking capabilities, the time period can be determined based on the gaze of the patient character in relation to the virtual environment. If the patient character looks down into the floor when asked to order a coffee, the computer system can set a shorter time period to provide another prompt more quickly. Conversely, if the patient character maintains eye contact with a task character (e.g. the barista in the café scenario) then the time period may be extended.

In another example, the proximity sensor is used to determine the patient character's distance to an object or virtual character in a scenario and to determine or adjust the time period based on this distance. For example, if the patient character approaches the task character (e.g. stepping forward towards the barista) then the time period may be extended, to give the patient more time to successfully complete the task before providing a prompt. If it is determined that the patient character is too far away or is moving further away, then the time period can be reduced to provide a prompt more quickly.

The time period may in general be based on the mental anxiety state parameter of the patient, which in turn can be based on eye gaze direction and/or proximity as well as other measurements.

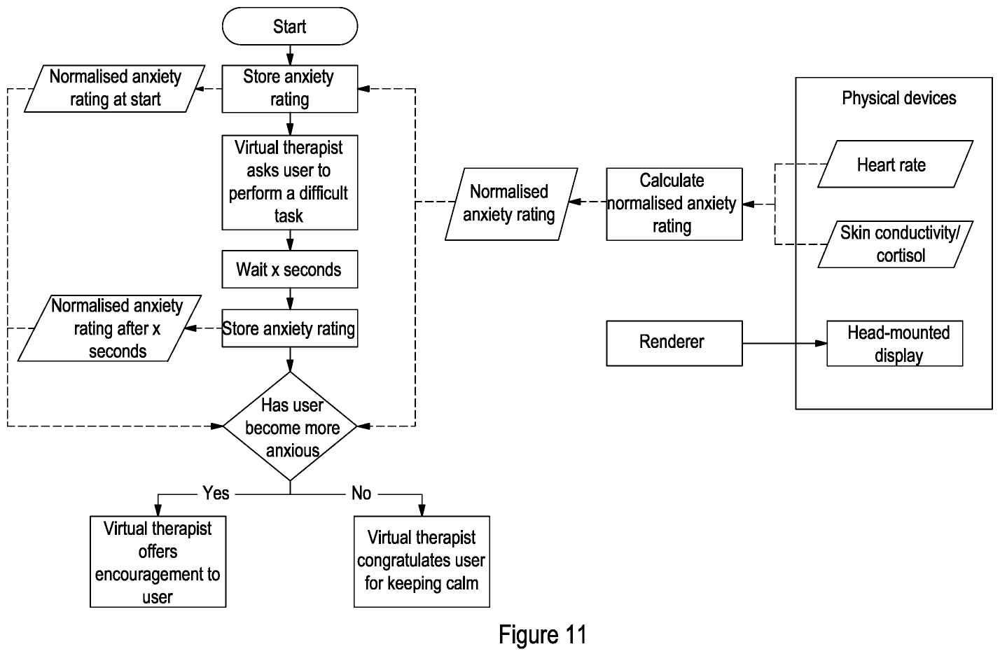

FIG. 11 is a flow diagram illustrating how the VR system or a patient mobile device can determine whether to provide a further prompt to the patient in a scenario or real world situation based on an anxiety rating determined from the patient's biological response. An anxiety rating associated with the patient is stored. The anxiety rating is based on the measured heart rate and skin conductivity (GSR) and/or cortisol level of the patient and is set as a normalised anxiety rate. The scenario or situation has an associated task action for the patient or patient character to complete, e.g. ordering a coffee. The coach asks the patient to perform the task action. After x seconds a new anxiety rating is calculated. The new anxiety rating is compared to the normalised anxiety rating to determine an action. If the anxiety rating has increased by more than a threshold value, a further prompt/encouragement is provided to the patient. If the state parameter indicates no significant increase in the anxiety rating, then no action is taken. After determining the new anxiety rating, this value can be set as the new normalised anxiety rating. The process can be reiterated until the required task action has been performed, or until a pre-set number of prompts have been provided. In a specific example, the anxiety rating is updated every second (i.e. x=1) in order to quickly determine if the patient is becoming more anxious. The anxiety rating is thereby used to dynamically set the time period between prompts provided as a function of the patient's anxiety.

In other examples, when in a VR session, instead of or in addition to calculating the anxiety rating based on the heart rate and skin conductivity, the anxiety rating can also be based on the patient character's actions in the virtual environment. For example, the anxiety rating can be based on the patient character's gaze using eye tracking.

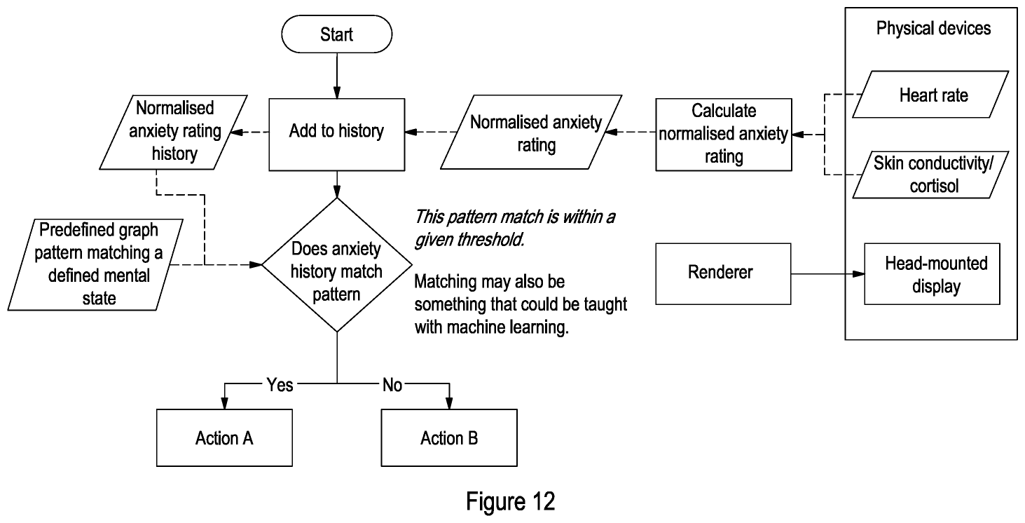

FIG. 12 is a flow diagram illustrating a more advanced method of determining when to provide a prompt to the patient character based on a mental anxiety state parameter. Instead of just comparing the current anxiety rating to a normalised anxiety rating, the normalised anxiety rating history is compared to a pre-defined pattern. This pattern can be based on data from previous sessions with the patient. Depending on if the anxiety history matches the pre-defined pattern, the coach can be used to provide appropriate feedback (e.g. a further prompt to complete the task action).

The cortisol/sweat level can be measured by including a wearable sweat biosensor. Users with anxiety tend to sweat on the VR hand controllers, and the biosensor can be included as stickers pasted to the hand controllers. The cortisol/seat level can also be used as a predictor of treatment needs (e.g. to determine an appropriate scenario/level). It may also be used to provide an objective outcome measure of treatment by comparing the levels of cortisol/sweat before and after completing a therapy session.

During a session, the following information may be logged:
General information: Participant ID, session number, date, and time, application build.
Tracking data: Head position and orientation, Hands position and orientation, controller button pressed, controller button released.
Scenario events: Scenario starts/ends and Scenario paused/resumed
User input/action/interaction with orbs and other similar props, and voice if applicable: Initial calibration (countdown), confidence, anxiety level, and similar questions that require an input from participant, start/continue/end session options, reset position, walk to the footprints.

The actions of the patient character within the VR environment that the VR system is able to detect include:
Café level 5—Pop bubble
Shop levels 3, 4—Pick up objects and drop objects.
Bus levels 1 to 5—Card payment
Bus level 4—Push bus stop button
GP level 3—Grab leaflets and drop leaflets.
GP level 4—Pick up pens and pass pens
GP level 5—Tell receptionist you have been waiting for a while?
Pub level 4—Pointing direction towards toilet?
Pub level 5—Ring last order bell
Street level 4—Press trigger to spray water and release trigger.

The interactions are designed to enable the key uptake of learning to experiment with thoughts relating to automatic responses to produce therapeutically relevant change. Prompts are designed at the critical points in the treatment where difficulty and anxiety is expected and are especially effective as they are delivered in a range of ways (by the coach, by other virtual characters, verbally, and visually) to increase the chance of successful learning.

Raw data can be collected during the VR session, which can be processed by the computer system to determine more useful data structures, such as:
How long did a patient character take to respond to a question?
How long did a patient character take to perform an activity?
How confident was the patient when performing an action, based on a model of upper limb motion?
How much time did a patient character spend looking down on the floor?
Did a patient character look directly at the eyes of other characters? How often?
How far away was the patient character from the virtual coach? Did the distance change over the sessions?
Did the patient character press the correct button? (a delay could be caused just because they are not familiar with the hand controllers).

The computer system can use such data structures as input, and based on the patient data determine the correct therapeutic scenario and difficulty level to provide therapy to that patient, for example using the neural network and predictor module. As more data is gathered over time, using the VR system it can be predicted how many sessions or which scenarios/difficulty levels a participant is recommended to pursue. This could be useful to help services manage patient treatments, resources for treatment (quota for taking in more patients), and estimating how many headsets are needed, costs etc. Machine learning can be used with the data to improve these predictions.

Patients and health services typically think in terms of particular and unique psychological disorders: depression, for example, or generalised anxiety disorder. However, people with one disorder are likely to also meet the criteria for other disorders—sometimes as many as three or more. It is also clear that psychological problems are dimensional. That is, they exist on a spectrum of severity. Depression, for example, is less a discrete category of experience than a relatively severe instance on a spectrum of low mood. Over the course of their life, most people will find themselves at different points on this spectrum. Moreover, many experts believe that mental disorders are not distinct disorders but complex combinations of psychological problems, which themselves are dimensional.

The above factors mean that there is significant scope for trans-diagnostic treatment techniques to complement disorder-specific interventions. Examples of the VR systems described herein can make such treatments available. For example, the VR system can implement different scenarios to help users become more active; to overcome worry; to understand the effect that thoughts can have on feelings; and to engage with everyday activities. These scenarios present evidence-based cognitive and behavioural therapeutic techniques. As such, they are likely to be of great value for people with a wide range of psychological problems including depression and anxiety, which are the most common disorders. We are pursuing a modular approach to treatment provision. The VR system provides a VR platform, which comprises a wide range of VR scenarios, each focused on a particular therapeutic technique, which may be supported by mobile applications and third party therapy tools. Many of these VR scenarios, or modules, will be trans-diagnostic. Others will be focused on particular disorders (for example, OCD). Specific combinations of modules can be tested to establish efficacy for particular diagnoses. The VR platform can provide a dashboard from which clinicians and patients can select the modules they believe will be most effective for them.

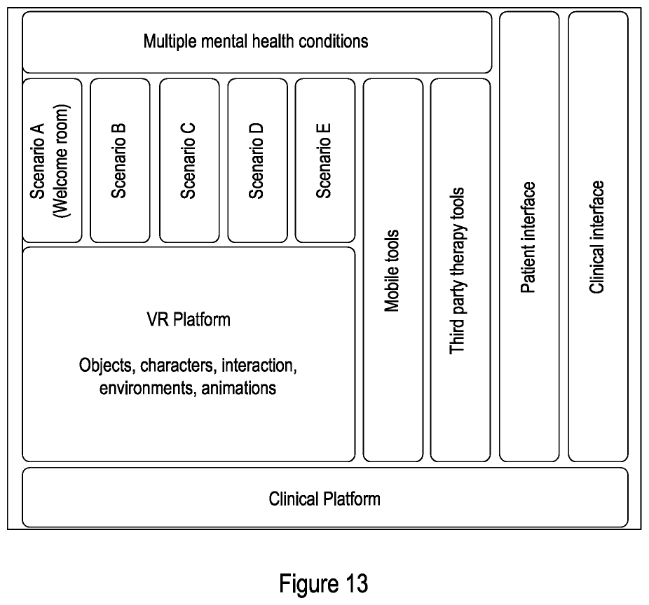

FIG. 13 shows a schematic diagram of how the VR platform can be as a part of a general clinical platform to treat multiple mental health conditions. The VR platform provides access to different scenarios (scenarios A to E), which can be used to treat one or more disorders.

Many alternatives will occur to the skilled person. The invention is not limited to the described embodiments and encompasses modifications apparent to those skilled in the art lying within the spirit and scope of the claims appended hereto.

1. A Virtual Reality (VR) therapeutic system for providing psychological therapy to a patient, the VR therapeutic system comprising:
a VR system to provide a virtual environment, the VR system including at least a head-mounted display unit with a sound generation capability and a VR input device; and
one or more computers coupled to or part of the VR system and configured to:
present a set of therapeutic scenarios to the patient in the virtual environment using the VR system, wherein each therapeutic scenario comprises at least one scenario task to be performed by the patient in the virtual environment by interacting with the VR system,
wherein the patient is represented by a patient character in the virtual environment, wherein at least one of the scenario tasks includes at least an interaction task in which the patient character interacts with a task character in the virtual environment or a non-verbal action task in which the patient character performs a non-verbal action in relation to one or more objects in the virtual environment, and wherein at least some of the time the virtual environment includes a representation of a coach character;
determine a mental anxiety state parameter representing a mental anxiety state of the patient by measuring, in the virtual environment, one or more characteristics of the interaction of the patient character with the task character or the one or more objects;
monitor performance of the scenario task in the virtual environment;
in response to one or both of non-performance of the scenario task and a state of patient mental anxiety indicated by the mental anxiety state parameter:
assist the patient in completion of the scenario task in the virtual environment by using the coach character to provide VR feedback comprising one or both of a verbal prompt and a visual prompt in the virtual environment.

2. A VR therapeutic system according to claim 1, wherein determining the mental anxiety state parameter comprises measuring, in the virtual environment, one or more of: eye gaze direction, proximity of the patient character to the task character, time to respond to a verbalization of the task character, time to act in relation to the one or more objects.

3. A VR therapeutic system according to claim 1, further comprising a patient state neural network having one or more inputs to receive the one or more characteristics of the interaction of the patient character with the task character or the one or more objects, and having an output to provide the mental anxiety state parameter representing the mental anxiety state of the patient.

4. A VR therapeutic system according to claim 1, wherein the feedback from the coach character includes speech, and wherein the VR system further comprises a patient mobile device configured to monitor one or both of patient activity and a measure of patient anxiety when the patient is in the real world and away from the virtual environment and to identify, in response to the monitoring, when the patient is in a stressful real world situation and, in response to the identifying, provide real world feedback when the patient is in the real world and away from the virtual environment, the real world feedback comprising a spoken audio output to the patient in the voice of the coach character in the virtual environment.

5. A VR therapeutic system according to claim 4, wherein the one or more computers are further configured to capture patient data from the patient whilst the patient is in the therapeutic scenario in the virtual environment, and wherein the patient mobile device is configured to use the captured data to provide the real world feedback.

6. A VR therapeutic system according to claim 4, wherein the patient mobile device comprises or consists of a wearable device to monitor the heart rate and/or galvanic skin response and/or cortisol level of the patient in the real world and away from the virtual environment, to determine the measure of patient anxiety.

7. A VR therapeutic system according to claim 1, wherein the set of therapeutic scenarios comprises a set of different therapeutic scenarios, each configured to simulate a real-world scenario the patient finds difficult, and each having a set of difficulty levels, wherein the one or more computers are further configured to provide a welcome room VR scenario including the coach character, and wherein within the welcome room the patient character is enabled to select one of the therapeutic scenarios and a difficulty level for the selected therapeutic scenario, wherein the difficulty level is not less than a previously completed difficulty level for the therapeutic scenario.

8. A VR therapeutic system according to claim 1, wherein the one or more computers are further configured to modify the selected therapeutic scenario to change the difficulty level by modifying one or more of: a background noise level, a number of task characters in the selected therapeutic scenario, a duration of the selected therapeutic scenario, a location of the task in the virtual environment, and a behavior of the task character(s).

9. A Virtual Reality (VR) therapeutic system for providing psychological therapy to a patient, the VR therapeutic system comprising:
VR hardware to provide a virtual environment, the VR hardware including at least a head-mounted display unit with a sound generation capability and a VR input device, and
a computer coupled to or part of the VR hardware; wherein the VR therapeutic system is configured to implement:
a VR environment module to present a set of therapeutic spatial regions to the patient in the virtual environment using the VR system, wherein each therapeutic spatial region comprises at least one patient action task to be performed by the patient in the virtual environment by interacting with the VR system, wherein the patient is represented by a patient character in the virtual environment, wherein at least one of the patient action tasks includes at least an interaction task in which the patient character interacts with a task character in the virtual environment or a non-verbal action task in which the patient character performs a non-verbal action in relation to one or more objects in the virtual environment, and wherein at least some of the time the virtual environment includes a representation of a coach character;
a mental anxiety measurement module to determine a mental anxiety state parameter representing a mental anxiety state of the patient by measuring, in the virtual environment, one or more characteristics of the interaction of the patient character with the task character or the one or more objects;
a performance monitoring module to monitor performance of the patient action task in the virtual environment;
a detector module to detect one or both of non-performance of the patient action task and a state of patient mental anxiety indicated by the mental anxiety state parameter; and
a patient feedback module to assist the patient in completion of the patient action task in the virtual environment by using the coach character to provide VR feedback comprising one or both of a verbal prompt and a visual prompt in the virtual environment in response to detection of the state of patient mental anxiety.

10. (canceled)

11. (canceled)

12. A Virtual Reality (VR) therapeutic system for providing psychological therapy to a patient, the VR therapeutic system comprising:
a computer system;
a head-mounted display unit for the patient, coupled to or comprising the computer system; and
one or more sensors for detecting patient actions performed by the patient, the sensor(s) being connected to the computer system,
wherein the computer system is configured to:
cause the display unit to display a virtual environment for a patient character, the virtual environment comprising a task character in a therapeutic scenario and, temporarily or permanently, a coach character;
cause the display unit to prompt the patient to perform a pre-defined action in relation to the task character;
determine with the sensor(s) whether the patient has performed the pre-defined action within a first time period;
in response to determining that the patient has not performed the pre-defined action within the first time period, cause the display unit to have the coach character provide an additional prompt for the patient to perform the pre-defined action.

13. A VR therapeutic system according to claim 12, wherein the computer system is further configured to:
after the additional prompt, determine whether the patient has performed the pre-defined action within a second time period;
in response to determining that the patient has performed the pre-defined action, cause the display unit to progress the virtual environment to a different therapeutic scenario in the virtual environment or to a next level of the therapeutic scenario; and
in response to determining that the patient has not performed the pre-defined action within the time period, initiate an exit sequence to exit the patient from the virtual environment.

14. A VR therapeutic system according to claim 12, further comprising a hand controller system to track the patient's hand movements, the hand controller system comprising a sensor of the one or more sensors, and wherein the pre-defined action comprises touching, holding or moving a virtual object in the virtual environment.

15. A VR therapeutic system according to claim 12, wherein the one or more sensors includes a microphone, and wherein the pre-defined action comprises speaking to the task character.

16. A VR therapeutic system according to claim 12, wherein the computer system is further configured to obtain one or more input parameters relating to behavior of the patient, and to adjust the first time period based on one or more of the input parameters,
wherein an input parameter of the one or more input parameters is dependent upon input received by the computer system from the one or more sensors, and
wherein the one or more sensors includes an eye tracking device mounted to the head-mounted display unit and wherein the input parameter is dependent upon time spent by the patient looking at a predefined region of the virtual environment.

17. (canceled)

18. (canceled)

19. (canceled)

20. (canceled)

21. A VR therapeutic system according to claim 12, wherein the computer system is configured to cause the display unit to progress the virtual environment to a next level of the therapeutic scenario in response to determining that the patient has performed the pre-defined action.

22. A VR therapeutic system according to claim 12, wherein the coach character interacts with the patient character in the virtual environment by speaking to the patient character to provide the additional prompt, the VR therapeutic system further comprising a patient mobile device, wherein the patient mobile device has a patient sensor input to sense an anxiety level of the patient and an audio output, and wherein the mobile device is configured to process data from the patient sensor input to monitor an anxiety level of the patient when the patient is in the real world and away from the virtual environment and to provide an audio output in response to the detected anxiety level, wherein the audio output comprises instructions to the patient in a voice of the coach character,
wherein the patient mobile device comprises or consists of a wearable device to monitor one or both of a heart rate and a galvanic skin response of the patient to sense the anxiety level of the patient, and wherein the audio output is provided in response to detection of an increased anxiety level.

23. (canceled)

24. A VR therapeutic system according to claim 12, further comprising a patient device and a remote server,
wherein the remote server is configured to receive patient data from the computer system collected while the patient is in the therapeutic scenario in the virtual environment, wherein the patient data represents performance of the patient in the virtual environment therapeutic scenario; and
wherein the patient device is configured to communicate with the remote server and to output data to the patient dependent upon the performance of the patient in the virtual environment therapeutic scenario to facilitate performance of the patient in a real world scenario corresponding to the virtual environment therapeutic scenario.

25. A VR therapeutic system according to claim 24,
wherein the patient device is configured to collect further patient data dependent upon the performance of the patient in the real world scenario corresponding to the virtual environment therapeutic scenario, and to transmit the further patient data to the computer system via the remote server; and
the remote server is further configured to adjust the virtual environment therapeutic scenario dependent upon the further patient data such that the virtual environment therapeutic scenario is adapted dependent upon the performance of the patient in the real world scenario.

26. (canceled)

27. A VR therapeutic system as claimed in claim 1, configured to collect patient data from the patient, wherein the patient data characterizes performance of the patient on the scenario task or pre-defined action, and further comprising a machine learning subsystem or module configured to process the patient data to determine a therapeutic scenario or level of therapeutic scenario or a number of therapeutic scenarios or levels of therapeutic scenario for presentation to the patient in the virtual environment.

28. (canceled)

29. A VR therapeutic system according to claim 3, wherein the patient state neural network comprises one or more initial input neural network layers configured to receive the neural network input and to generate a latent representation of the neural network input; at least one intermediate neural network layer having an intermediate layer input coupled to the one or more input neural network layers, and having an intermediate layer output; and one or more output layers having an input coupled to the intermediate layer output to generate the neural network output, wherein the neural network output defines a score distribution over a discrete set of possible patient anxiety values, and wherein the patient state neural network is configured to generate the mental anxiety state parameter from the score distribution.
